(19) United States
(12) Patent Application Publication
(10) Pub. No.: US 2019/0380646 A1
(43) Pub. Date: Dec. 19, 2019

(54) ELECTRONIC HEADWEAR

(71) Applicant: Oxystrap International, Inc., San Diego, CA (US)

(72) Inventors: Bruce Gertsch, San Diego, CA (US); Ronald Gertsch, San Diego, CA (US); Paul Nysen, Pala, CA (US); Peter Nysen, San Jose, CA (US); William Swanson, San Diego, CA (US); Christopher L. Gehrisch, Vista, CA (US); Martin D. McCune, San Diego, CA (US); David L. Williams, San Diego, CA (US)

(73) Assignee: Oxystrap International, Inc., San Diego, CA (US)

(21) Appl. No.: 16/362,569

(22) Filed: Mar. 22, 2019

Related U.S. Application Data
(63) Continuation-in-part of application No. 13/853,526, filed on Mar. 29, 2013, now Pat. No. 10,265,019.

(51) Int. Cl. A61B 5/00; A61B 5/1455; A41D 20/00; A61B 5/0205; A61B 5/01

(52) U.S. Cl. CPC A61B 5/6803; A61B 5/14552; A61B 5/0004; A41D 20/00; A61B 5/0205; A61B 5/021; A61B 2560/0214; A61B 2562/185; A61B 2562/0238; A41B 2400/60; A41D 2400/82; A61B 5/01

(57) ABSTRACT

A headwear assembly is disclosed having an oximetry sensor and a circuit assembly configured to measure pulse and oxygen level for a user during exercise and/or physical activity. The oximetry sensor is positioned in a front section of the headwear assembly, proximate to the forehead when worn. Other sensors can also be included with the headwear assembly, such as temperature, blood pressure, and so on. The headwear assembly is configured to securely conform about a user's head when worn, such that the oximetry sensor is positioned and effectively immobilized on the forehead above the eyebrows. The headwear assembly can further provide integrated functionality with an external electronic device, such as a smart mobile phone. The headwear assembly can include a wireless charger for charging a rechargeable battery disposed on the headwear assembly.

### CROSS-REFERENCE TO RELATED APPLICATION

This application is a continuation-in-part of U.S. patent application Ser. No. 13/853,526, filed Mar. 29, 2013, and which is incorporated by reference.

### FIELD OF THE INVENTION

The present invention relates generally to athletic headwear and, more particularly, headwear having a variety of physiological sensors, such as oxygen saturation, body temperature, pulse rate, and blood pressure.

### BACKGROUND OF THE INVENTION

There has been an increasing interest in using devices and tools during exercise and athletic activities to enhance performance and also to monitor critical conditions. Measuring physiological characteristics during exercises can optimize workout routines. One such tool is an oximetry unit, which measures the oxygen saturation in blood and pulse rate. During exercise and at higher altitudes, blood oxygen levels may drop. When the body is deprived of an adequate supply of oxygen, tissue hypoxia may occur. Therefore, monitoring blood oxygen levels with an oximetry unit can be used to guide exercise, athletic training, and provide an alert in critical conditions and situations.

Oximetry is a noninvasive assessment of arterial oxygen saturation (SpO2), which is the measurement of the amount of oxygen carried by hemoglobin in the blood stream. It relies on Beer-Lambert's law, which states that the concentration of an absorbing substance in a solution is related to the intensity of light transmitted through that solution. Accordingly, an oximetry unit uses small light-emitting diodes (LED) to transmit light and then measures the light not absorbed by the tissue by a photodetector to determine the concentration of oxygen in blood. An oximetry unit emits light of at least two different wavelengths, red (660 nm) and infrared (905, 910, 940 nm). Deoxyhemoglobin (hemoglobin not combined with oxygen) has a higher optical extinction in the red region of the light spectrum compared to oxyhemoglobin (hemoglobin that is combined with oxygen). In contrast, in the infrared region, the optical absorption of deoxyhemoglobin is lower than oxyhemoglobin. Thus, based on the differences in light absorption, an oximetry unit can measure the amount of light absorbed to calculate the percentage of oxygen saturation in blood.

The oximetry sensor is usually placed on a thin part of the body such as a fingertip or an earlobe. Since oximetry sensors have been predominantly used for clinical or medical purposes, the site of the oximetry sensor placement has generally not been an issue because multiple satisfactory placement sites are readily available. However, during exercise or other athletic activities, traditional locations for oximetry sensor placement such as fingertip or earlobe can be problematic.

It should be appreciated that there remains a need for an assembly that easily measures physiological vital sign changes such as oxygen saturation, pulse, body temperature, and blood pressure of a user during physical exercise, athletic activities, and other critical situations. The present invention addresses this need and others.

### SUMMARY OF THE INVENTION

Briefly, and in general terms, the invention provides an electronic headwear assembly, such as a self-contained electronic strap, that measures real-time physiological changes, e.g., oxygen saturation, pulse, blood pressure, and body temperature of a user during physical activity and other critical situations. The headwear assembly is configured to securely conform about a user's head when worn, such that the oximetry sensor is positioned and effectively immobilized on the forehead above the eyebrows. The headwear can include an electronic flex circuit comprising a sensing and circuit assembly, which is housed in a waterproof/water-resistant casing. As such, the headwear assembly enables the measurement of a user's oxygen level and other physiological parameters while exercising or performing physical activities.

In various embodiments in accordance with the invention, the headwear assembly can provide: integrated functionality with multitudes of functions of an external device such as a smart mobile device via wireless Bluetooth® or Wi-Fi transceivers (e.g., to announce, display and record real-time physiological and other data). The external device may have either wired or wireless connection for listening or listening may occur via the speaker of the external mobile device.

When placed in a headwear assembly, a preferred location for the oximetry sensor is in the front section and proximate to the forehead when worn. In an exemplary embodiment, the headwear assembly is a self-contained unit that is housed in a flexible material with a plurality of attachment means, such as hook-and-loop fasteners, for adjustability and attachment. The front of the headwear assembly can be marked to indicate the proper position of the oximetry sensor, so that the user can ensure that the oximetry sensor is in the appropriate location on the user's forehead.

In an exemplary embodiment, the headwear assembly includes an oximetry sensor, and a flexible circuit board assembly that is separately spaced apart from and electrically coupled to the oximetry sensor by a flexstrip, or other means. More specifically, the oximetry sensor and circuit board assembly are confined in a flexible and transparent waterproof and/or water-resistant casing, which not only protects against moisture, but also does not interfere with oximetry measurements due to its transparency. Additionally, unlike non-flexible waterproof casings, the flexible nature of the waterproof casing used for the current invention avoids placing mechanical stress on the flex circuit trace wiring despite repeated bending of the flex circuit during usage.

In a detailed aspect of an exemplary embodiment, the present invention provides an oximetry sensor, having an LED and photodetector sensor, which measures percentage of oxygen saturation, and pulse rate. Additionally, a temperature sensor can be provided.

In another detailed aspect of an exemplary embodiment, the circuit board assembly includes an LED driver circuit, a detector circuit, a processor, and battery. The circuit board assembly is programmable and syncs with an external smart mobile Bluetooth compatible device to provide physiological data, such as body temperature, blood pressure, oxygen saturation, pulse and other data to the user. In another aspect, audio prompts are announced periodically by way of the listening device to the user at preprogrammed levels.

In yet another detailed aspect of an exemplary embodiment, the circuit board assembly includes a wireless transceiver. In this manner, physiological data can be transmitted wirelessly to a recorder and display unit, such as a smart mobile device, that could be worn, for example, on the arm, wrist, and other part of the body. Furthermore, the circuit board assembly can be configured to continuously measure the user's physiological characteristics and wirelessly transmit data to the synced or connected smart external mobile device.

In yet another exemplary embodiment, a headwear assembly can include a self-contained oximetry electronic flex circuit, which defines a section for a sensor module, an analog module, a processor module, and a battery module. The flex circuit can be configured without any external openings or physical connections, and can be enclosed within a headwear material, except for an aperture for the sensor module. The electronic flex circuit can include a flex circuit material substrate (flexible substrate) wherein the electronic components are mounted thereon. Moreover, the flexible substrate can include stiffeners for protection of the electronic components. The stiffeners are disposed on a side of the flexible substrate that is opposite to a corresponding module, and are coupled to one another via said flexible substrate, thereby enabling the flex circuit to conform to the user's head shape when the headwear assembly is worn. Moreover, the flexible substrate can include peripheral “wing” extensions which can secure the flex circuit to the headwear assembly by sewing, RF sealing (Radio Frequency) with vinyl material interface, as well as other methods.

In another detailed embodiment of the various headwear embodiments, a headwear can also include slip-resistant bands disposed about the interior surface, such that the bands can grip the hair and skin. For the headwear assembly having the electronic flex circuit, the combination of slip-resistant bands, the securing mechanism of the headwear via the wings of the flexible substrate, the use of elasticized headwear material, and an adjustable attachment assembly provides precise tension and immobilization for the headwear when worn. As such, this enables to maintain the sensor module positioning for physiological data accuracy, especially during movement during physical activity.

In a detailed embodiment of the various headwear embodiments, the headwear can include a wireless charger configured to charge a rechargeable battery disposed in the battery module via inductive charging. The wireless charger can be secured to the headwear assembly via a charging pocket located on the interior side of the headwear, and aligned with the battery module.

For purposes of summarizing the invention and the advantages achieved or implemented over the prior art, certain advantages of the invention have been described herein. Of course, it is to be understood that not necessarily all such advantages may be achieved or implemented in accordance with any particular embodiment of the invention. Thus, for example, those skilled in the art will recognize that the invention may be embodied or carried out in a manner that achieves, optimizes, or implements one advantage or group of advantages as taught herein without necessarily achieving or implementing other advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment disclosed.

### BRIEF DESCRIPTION OF THE DRAWINGS

Embodiments of the present invention will now be described, by way of example only, with reference to the following drawings in which:

FIG. 1A is a perspective view of a headwear assembly in accordance with the present invention, depicting sensors and a circuit board assembly that are spaced apart and electrically coupled together.

FIG. 1B is an elevational view depicting a user wearing the headwear assembly of FIG. 1A, further wearing a smart mobile device housed in an armband and wireless earphones. The wireless earphones, the headwear assembly, and the smart mobile device are wirelessly and operatively connected.

FIG. 1C is a simplified block diagram of the headwear assembly, smart mobile device, and earphones of FIG. 1B.

FIG. 2 is a simplified block diagram of the oximetry sensors and the circuit assembly of the headwear assembly of FIG. 1A.

FIG. 3 is a plan view of a headwear assembly in accordance with the present invention, depicting the front surface of a headband (facing away from a user's head), laid open, including an attachment mechanism for coupling opposing ends of the headband together.

FIG. 4 is a plan view of a second embodiment of a headwear assembly in accordance with the invention, depicting an interior surface of a headband (facing towards a user's head), laid open, having an oximetry electronic flex circuit with a sensor area and transparent window exposed through a front aperture of the headband, and a plurality of slip-resistant bands disposed about the headband.

FIG. 5 is a detailed block diagram view of the electronic flex circuit of FIG. 4.

FIG. 6A is a simplified block diagram of the electronic flex circuit of FIG. 4 depicting the various modules present.

FIG. 6B is a front view of the electronic flex circuit of FIG. 4, that faces towards the forehead of a user when the headwear assembly is worn, depicting the transparent window present in the sensing module.

FIG. 6C is a rear view of the electronic flex circuit of FIG. 4, that faces away from the forehead of a user when the headwear assembly is worn depicting the battery module, processor module, and analog circuit module.

FIG. 6D is an elevational view of the electronic flex circuit of FIG. 4.

FIG. 7 is a top view of the rear side of the electronic flex circuit of FIG. 4, and further depicts a corresponding wireless charger.

FIG. 8 is a plan view of the headwear assembly of FIG. 4, further depicting a charging pocket and a wireless charger inserted therein.

FIG. 9 is a perspective view of a sensor cover for the sensor module disposed on the headwear assembly of FIG. 4.

FIG. 10 is a perspective view of a headband configured with the headwear assembly of FIG. 4, disposed about a user's head, and having light barriers.

FIG. 11 is a perspective view of the electronic flex circuit of FIG. 4, depicting a battery cap and processor cap that can be disposed on the respective module.

FIG. 12 is a plan view of a third embodiment of a headwear assembly in accordance with the invention, depicting an interior side of a headband, laid open, having multiple sensing sections exposed through apertures of the headband.

FIG. 13 is a simplified block diagram view of the sensors and the circuit assembly of the headwear assembly of FIG. 12.

FIG. 14 is a simplified block diagram view of sensors and the circuit assembly of another embodiment of a headwear assembly in accordance with the invention.

FIG. 15 is a simplified block diagram view of sensors and the circuit assembly of another embodiment of a headwear assembly in accordance with the invention.

FIG. 16 is a cross-sectional view of a blood pressure sensor for use in selected embodiments of a headwear assembly in accordance with the invention.

FIG. 17A is a front view of the electronic flex circuit as shown in FIG. 6B, identifying the stiffeners and wing extensions located on the flexible substrate.

FIG. 17B is a rear view of the electronic flex circuit as shown in FIG. 6C, identifying the flexible substrate and neck regions between sections.

FIG. 18A is a front view of the electronic flex circuit as shown in FIG. 6B, identifying the waterproof/water-resistant casing enclosing the flex circuit.

FIG. 18B is a rear view of the electronic flex circuit as shown in FIG. 6C, identifying the waterproof/water-resistant casing enclosing the flex circuit.

### DETAILED DESCRIPTION OF THE PREFERRED EMBODIMENTS

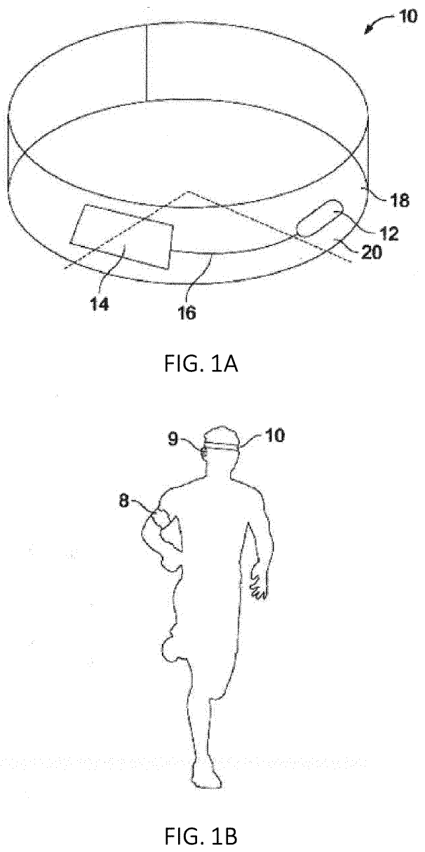

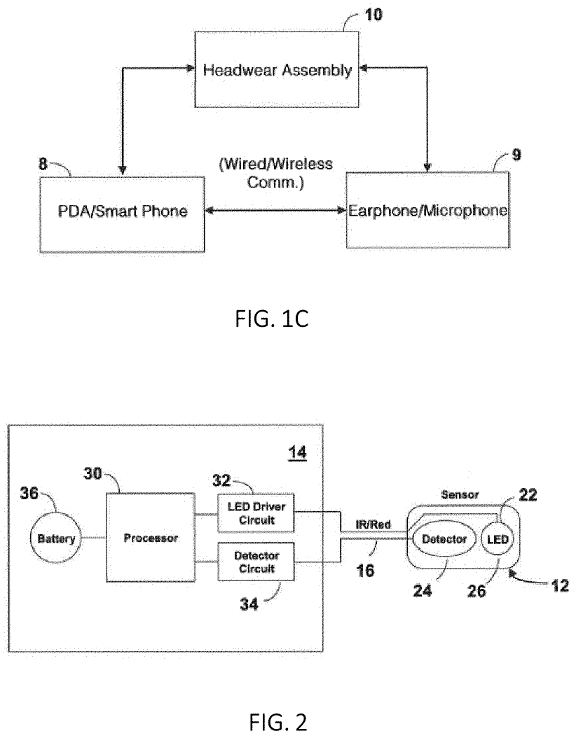

Referring now to the drawings, and particularly FIGS. 1A-1C, there is shown a flexible, non-disposable, non-medical and wearable headwear assembly 10 comprising a headband 18 having an oximetry sensor (emitter/detector) 12, and a circuit board assembly 14 that is spaced apart from and electrically coupled to the oximetry sensor (emitter/detector) 12 by a flexstrip 16, an air-tube, or other means. The oximetry emitter/detector 12 is located in a front section 20 of the headband, proximate to the forehead when worn. Other sensors can also be included with the headband, such as temperature, blood pressure, and so on. The headwear assembly 10 is configured to securely conform about a user's head when worn, such that the oximetry sensor is positioned and effectively immobilized on the forehead above the eyebrows. As such, the headwear assembly enables the measurement of a user's oxygen level, pulse, body temperature and blood pressure while exercising or performing physical activities.

The position of the sensor 12 on the head is advantageous for measuring accurate body vital signs during physical activity over peripheral areas, such as the fingertip, wrist, earlobe or other body locations. The head provides the ideal body location for optimal reflective oximetry (which is required for accuracy of physiological data during physical activity) since the head has optimal superficial blood supply and a good reflective surface. Moreover, it is the best body location for minimizing movement of the sensors during physical activities since it is a less active site compared to other body locations. It is not possible to receive optimal oximetry data on any other part of the body during physical activity because of the inability to obtain the required secure sensor immobilization plus a good superficial blood supply. Additionally, a person engaged in certain sports, such as tennis, cycling, running, and basketball, can be already accustomed to wearing headwear. The head is also less impacted by adverse conditions such as a cold environmental temperature, dehydration, and low blood pressure. By contrast, such conditions can substantially interfere with the blood flow in the peripheral areas of the body due to vasoconstriction.

Referring to the drawings in FIGS. 1B and 1C, the headwear assembly 10 can operate with an external smart mobile device 8, and a headset or earphone microphone system (9). These may communicate either directly by wire/cable or by wireless means. Thus, the headwear assembly can also be configured to provide other electronic functions, such as wireless communications (e.g., Bluetooth® and Wi-Fi transceivers), and other real-time data (e.g., altitude, average speed, steps, distance, time, calories burned and battery charge), plus mobile phone and radio/music player features, in addition to oxygen saturation, pulse, body temperature, and blood pressure.

Due to the natural placement of a headband during exercise, the oximetry sensor 12 will be located optimally on the mid-forehead above the eyebrows. When properly worn in the preferred location, the LED emitter 22 and detector 24 (FIG. 2) of the oximetry sensor 12 lie on the mid-forehead above the eyebrows in the flattest area of the forehead, allowing for good sensor skin contact, and also in a region of dense surface blood flow. The front 20 of the headband can be marked with the location of the sensor so the user can position the oximetry sensor in the appropriate location on the user's forehead.

In one embodiment, the headband 18 is made of a fabric that is wicking, stretchable, and breathable material, e.g., such as Dryline or spandex. More specifically, the headband can be made of wicking material to function as an effective sweatband, thereby preventing sweat from moisturizing the skin and hair underneath the headband, which can lead to slippage and movement of the headband and optical sensors. The fabric can also be antimicrobial with silver impregnated particles embedded within the fabric for odor control and stain resistance.

The sensors 12 and circuit board 14 are confined in a flexible waterproof casing that is transparent. In an exemplary embodiment, the sensors and circuit board are arcuate to conform comfortably to the user's head. Also, the sensors and circuit board are mounted on a flexible material that will conform to the user's head when worn. Furthermore, the wire (or air-tube) assembly 16 that connects the sensors and circuit board may include a waterproof and/or water resistant material, or is confined in the waterproof and/or water-resistant casing, and is flexible to conform to the user's head when the headwear assembly is worn.

With reference to FIG. 2, the oximetry sensor 12 includes the emitter (LED) 22 and the detector 24 covered by a flexible and transparent, waterproof and/or water-resistant material 26. The LED and detector are placed adjacent to each other.

In the exemplary embodiment, the LED 22 emits at least two distinct wavelengths of light: red and infrared light. The detector is a photodetector capable of detecting the wavelengths of light emitted by the LED.

More particularly, the LED 22 emits light at two wavelengths (e.g., (1) 660 nm (red light); (2) 905, 910, or 940 nm (infrared light)). As light passes through tissue, oxyhemoglobin absorbs infrared light and allows red light to pass through, while deoxyhemoglobin does the opposite and absorbs red light but allows infrared light to pass through. Via the detector 24, the assembly measures the absorption ratio of the red and infrared light. The percentage of oxygen saturation is then calculated. To calculate the ratio of oxygen saturation, by a means known to those versed in the art, as blood pulses and fades with each heartbeat, the measurement of oxygen absorption from the peak level of the pulse is subtracted from the measurement of oxygen absorption at the lower level. In other embodiments, additional or alternative approaches can be used to measure pulse rate, e.g., such as utilizing blood pressure sensors as discussed herein below.

With continued reference to FIG. 2, the circuit board assembly 14 includes a processor 30, LED driver circuit 32, detector circuit 34, and battery 36. The circuit board assembly is connected to the sensor assembly 12. The processor 30 connects to the LED 22 through the LED driver circuit 32. After instructions to the LED 22 have been sent, the processor 30 receives the light absorption measurement data from the detector 24, through the detector circuit 34. The battery 36 powers the circuit assembly 14 and the sensors 12. A rechargeable battery is used, and can be charged remotely using an inductive (magnetic) coupling method.

In a detailed aspect of an exemplary embodiment, the circuit board 14 contains memory that is coupled to the processor 30, making it programmable to provide customizable data to the user, which includes body temperature, oxygen saturation, pulse, blood pressure, battery level, and other data. In another aspect, the headwear assembly can be programmed to announce periodic audio prompts by way of a speaker at preprogrammed intervals or indirectly via an ear piece/microphone connection, either wired or wirelessly connected.

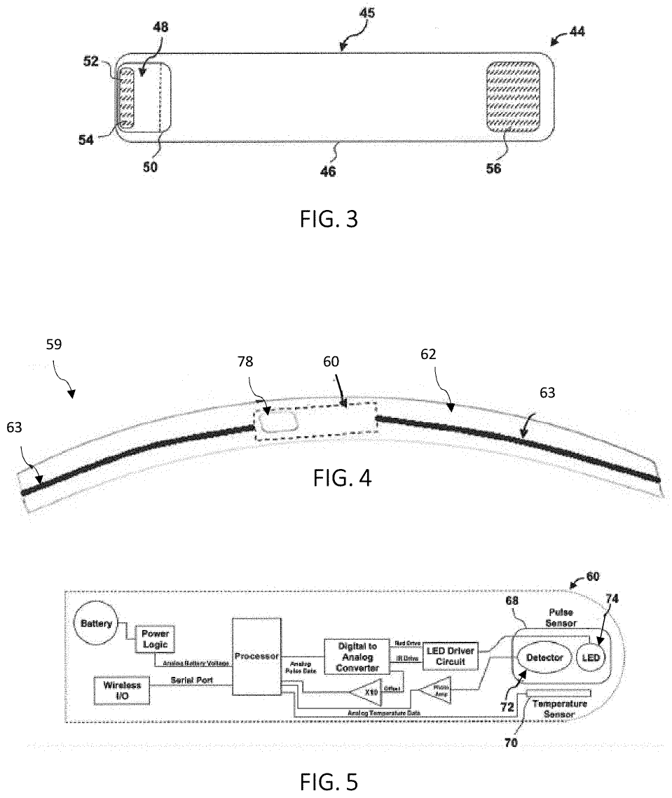

With reference now to FIG. 3, a front surface 46 of another embodiment of a headwear assembly 44 is shown. The headwear assembly 44 includes a headband 45 having an attachment tab 48 having a proximal portion 50 coupled to the front surface and a free distal end 52. An attachment tab includes a first portion 54 of an attachment assembly that mates with a corresponding second portion 56 of the attachment assembly disposed on an opposing end of the headband. The attachment assembly can be any number of those known in the art, e.g., hook and loop, snaps, fasteners, and other means. The attachment tab 48 provides adjustable tension to securely attach to the user's head during exercise or athletic activities.

With reference now to FIG. 4, another exemplary embodiment for a headwear assembly 59 is shown, depicting the interior surface that will be in contact with the head of a user. The headwear 59 is comprised of headwear material 62 having similar properties as aforementioned for other headwear embodiments, including wicking material. Moreover, the headwear 59 can be elasticized, which helps provide precise tension about a user's head when worn, thereby maintaining sensor immobilization and accuracy of physiological data. The headwear 59 can be equipped with slip-resistant bands 63 that are disposed about the interior surface of the headwear 59, and thus in contact not only with the skin of a user, but the hair as well. The slip-resistant bands 63 can be formed of polymeric material, e.g., silicone, which can be embedded into the headwear material and grip both the skin and hair. The slip-resistant bands 63 further enable improved immobilization of the headwear, specifically the sensor, wherein such improvement is present even in moisture environments. The headwear 59 can also include any of the aforementioned securing mechanisms, e.g., attachment assembly using hook and loop fasteners, snaps, and so on, enabling it to be secured about the head of a user. Maintaining proper tension of the headband on the head of a user avoids measurement inaccuracies, such as compromised blood flow with excess tension, or movement of the optical sensors if inadequate tension.

With reference to FIGS. 4-6, the headwear 59 can include an oximetry electronic flex circuit 60, which can comprise a battery module 61, a processor module 65 that can include a Bluetooth receiver, an analog circuit module 67, and a sensor module 69. The components of the electronic flex circuit 60 can operate similar to as described previously for the other circuit embodiments, e.g. sensor 12 and circuit board 14 (FIGS. 1-2), including having a LED driver and detector circuit, and wireless connection to an electronic device for announcing, recording, and displaying data. For example, FIG. 5 shows a block flow diagram for a rear view of the electronic flex circuit 60, which can include a pulse and oximetry sensor 68 having a detector 72 and LED 74, wherein the oximetry sensor is connected to a LED driver circuit, which communicates with a processor via a digital to analog convertor (D/A convertor). The detector 72 communicates with the processor via a photo amplifier. Moreover, other types of sensors can be included, such as a temperature sensor 70 (e.g., thermistor), which can be located adjacent to the sensor emitter/detector 68. The flex circuit can be configured to communicate bi-directionally with other devices via wireless communications, e.g., Bluetooth®, WI-FI, or inductive means.

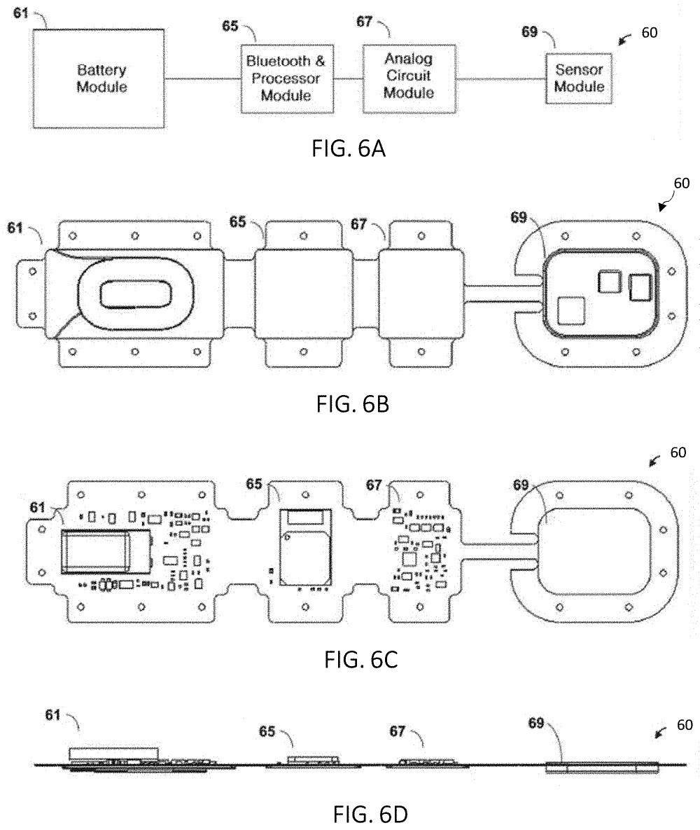

With reference now to FIGS. 6A-6D, an electronic flex circuit 60 is shown, configured to facilitate comfort and flexibility when worn against the head of a user, with the sensing end positioned to be disposed in an appropriate location for accurate measurements when worn. Components can be distributed about several circuit boards connected via bridge connections (e.g., flex wire, thin bridge of circuit board).

As seen in FIG. 6A and as aforementioned, the flex circuit 60 can include a battery module 61, communications (e.g., Bluetooth WIFI) and processor module 65, analog circuit module (analog) 67, and a sensor module 69. FIG. 6B represents the front side of a flex circuit 60, which faces towards the forehead of a user wearing the headwear 59, while FIG. 6C depicts the rear side of a flex circuit 60. FIG. 6D depicts an elevational view of the flex circuit 60.

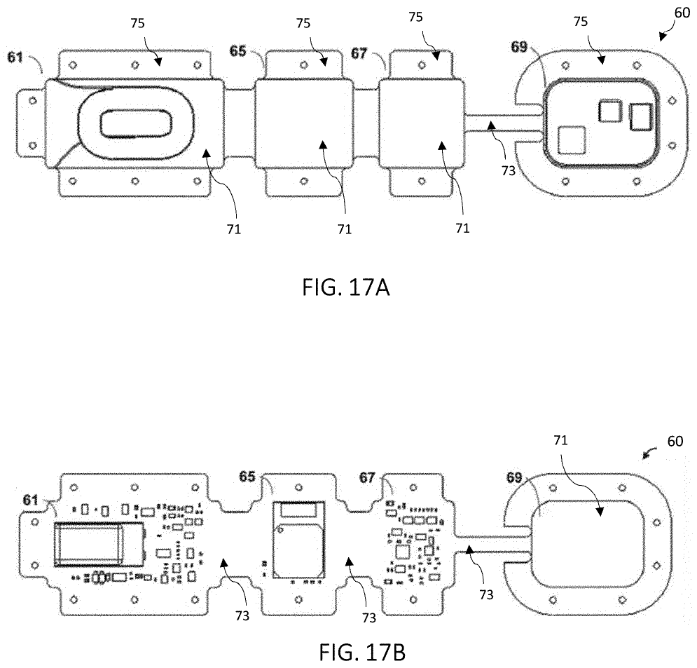

With reference now to FIGS. 6B-D and FIGS. 17A-B, the components of the electronic flex circuit 60, i.e. modules for battery, processor, analog, and sensor, are mounted on a flex circuit material substrate (“flexsubstrate”) 73, which is a flexible PCB (printed circuit board) material that can conform to the head of a user wearing the headwear assembly. The flexsubstrate 73 can be formed of a unitary construction. Moreover, the flexsubstrate can define separate sections for each module, with each section defining a neck region in between. Protective stiffeners 71 can be mounted to each section of the flexsubstrate 73, and be disposed on a side of the flexsubstrate that is opposite to a respective module. For example, the respective stiffeners 71 corresponding to the battery, processor, and analog modules will be disposed on a front side of the flexsubstrate 73, i.e. front side of the flex circuit 60 (FIG. 17A), while the stiffener 71 for the sensor module will be disposed on a rear side of the flexsubstrate 73 (FIG. 17B). By contrast, the battery 61, processor 65, and analog modules 67 are disposed on a rear side of the flexsubstrate 73, while the sensor module 69 is disposed on a front side of the flexsubstrate 73. These stiffeners provide additional robustness to help prevent said components (modules) from being damaged. The stiffeners 71 are coupled to one another via said flex circuit substrate 73, thereby enabling the flex circuit 60 to conform to the shape of a head that is wearing the headwear 59. Moreover, trace wiring included between the sections on the flexsubstrate 73 are configured to be flexible such that it can conform to the shape of a headwear 59 disposed about the head of a user. The flex circuit substrate 73 can also include peripheral “wing” extensions 75 that extend from each section, and enable the flex circuit 60 to be secured to the headwear 59. The “wing” extensions 75 can include apertures. Means of securing the electronic flex circuit 60, via the wing extensions 75, to the headwear 59, include sewing, RF sealing with vinyl interface, and so on. As such, the securing mechanisms via the “wing” extensions of the flexsubstrate 73 to the headwear embodiment enable the flex circuit 60 to maintain positioning on the head, thereby assuring complete immobilization.

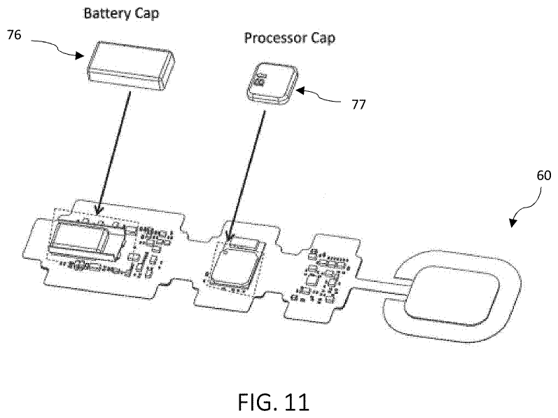

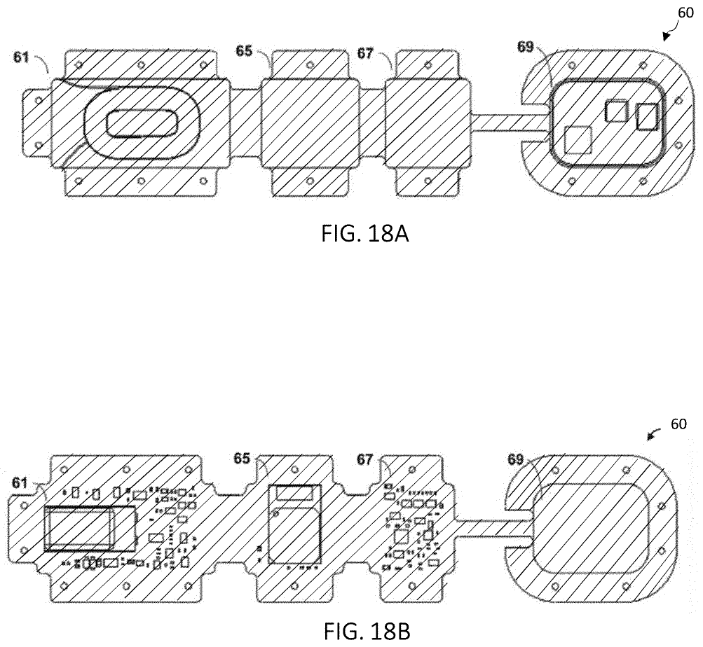

With reference to FIGS. 18A-B, the flex circuit 60 can include a flexible, transparent, waterproof and/or water-resistant casing, which can be sprayed on, manually applied, and so on. The waterproof casing, represented using hashed lines in FIGS. 18A-B, will enclose the entire flex circuit 60. The waterproof casing is transparent and thus does not interfere with oximetry data measurements via the optical sensors. Also, the casing does not place any mechanical stress on the electronic wiring of the flex circuit 60, which could otherwise cause damage to the wiring due to repeated bending during usage. Moreover, the casing does not leave any air pockets, which would expand at higher altitudes and likely result in the flex circuit 60 malfunctioning. Additionally, as seen in FIG. 11, the flex circuit 60 can be protected by two waterproofing barrier caps (76, 77), specifically protecting the battery and processor from moisture.

With reference now to FIG. 4, the flex circuit 60 can be enclosed by the headwear material 62 except for an aperture 78 disposed about the sensor module 69 to enable pulse and oxygen saturation measurement. Thus, the battery module 61, processor module 65, and analog module 67 are not visible to the user as they are enclosed by the headwear material 62. Within the aperture 78 on the headwear material is included a flat transparent window that covers the sensor module 69, thereby protecting the components in the sensor module 69, but also still allowing measurement of the pulse and oxygen saturation via the exposed skin. The transparent window can be easily cleaned, to ensure accuracy of the sensor measurements. As such, all the components of the electronic flex circuit 60 are self-contained with no external openings or ports, and no other external physical connections, including no battery charging port (as further described below). This is particularly beneficial for maintaining and enhancing waterproofness or water-resistance, as it eliminates any access points through which water can penetrate. Moreover, the flex circuit 60 can be configured without an on/off control button, such that the flex circuit 60 can be configured to be always powered on, and further be configured to automatically switch to a sleep mode when not actively used, thereby reducing power consumption and avoiding power drain.

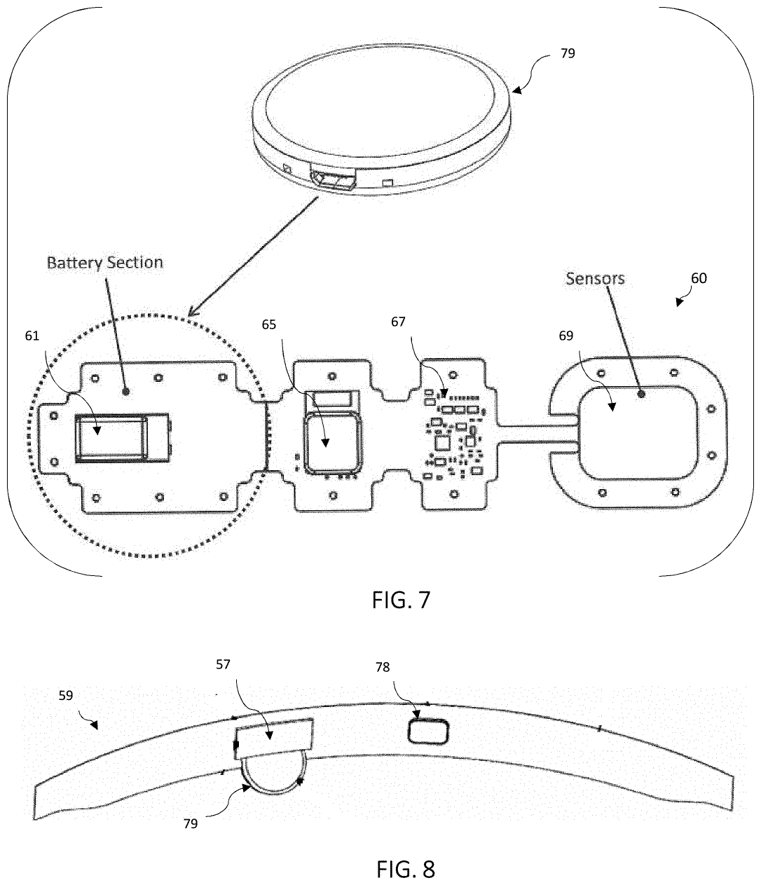

With reference now to FIGS. 7-8, and as aforementioned, the flex circuit 60 can be configured without a battery charging port by using a wireless inductive charger 79. The headwear 59 can include a non-stretchable cloth material that can act as a charger pocket 57, enabling a wireless charger 79 to be securely positioned so as to facilitate inductive charging of a rechargeable battery located in the battery module. The charger pocket 57 can be positioned to align the wireless battery charger 79 with the location of the battery module 61, separated by the headwear material 62 and waterproof casing.

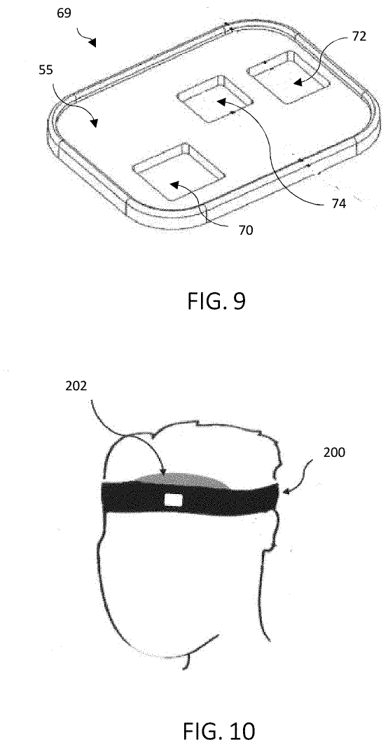

With reference now to FIG. 9, the sensor module 69 can include a sensor cover barrier 55 disposed between the emitter 74 and detector 72 of an oximetry sensor, thereby preventing light from passing from the emitter 74 to the detector 72 directly without first passing through the body tissues. Moreover, with reference now to FIG. 10, the headwear 59, embodied as headband 200 as an example, can be further equipped with expanded light barriers, so as to prevent inaccuracy of physiological data measured due to any ambient light, i.e. outdoor sunlight, etc. reaching the forehead near the oximetry sensor and interfering with sensor operation. The headband 200 can include an upper light barrier 202, a lower light barrier, and one or more side light barriers, such that the light barriers entirely surround the oximetry sensors for an adequate distance above, below and on both sides of the sensors. The light barriers can be made from a soft, flexible material and can be attached to the headwear embodiment. The upper barrier 202 can be configured to be aligned with the upper forehead, while the lower barrier can be configured to be disposed just above the eyebrows. The upper barrier, lower barrier, and side barriers cover the entire flex circuit, except for the front side of the sensor area itself, and can extend around the headwear to the sides of a user's head. The lower barrier and side barriers are enclosed by the headwear material 62.

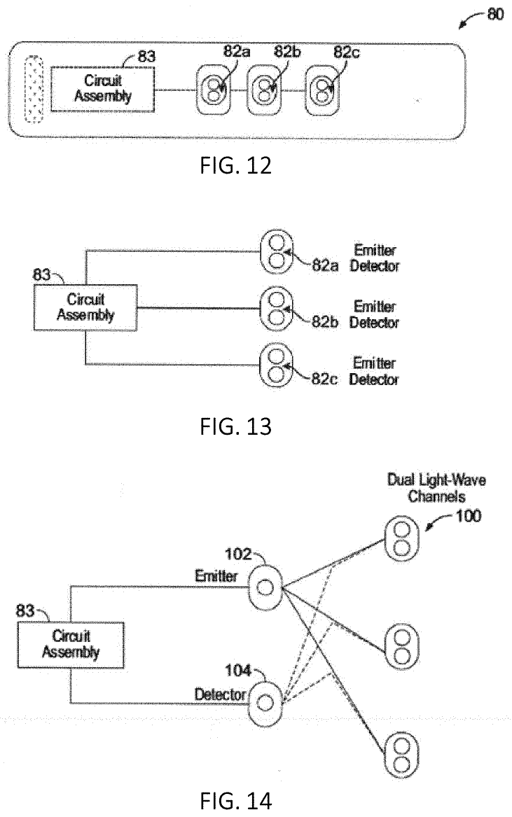

With reference now to FIGS. 12 to 15, an exemplary headwear assembly 80 can include a plurality of sensors 82 (a,b,c) for oximetry and/or for blood pressure (e.g., 90 of FIG. 16), in which such sensors are disposed in spaced relationship in prescribed locations about the headwear assembly to measure various selected locations on the user, when worn. The advantage of multiple sensors is that the unit can obtain the strongest and most accurate reading from multiple readings.

With reference now to FIG. 13, multiple sensors may be electronically or optically connected back to the circuit assembly 83. When optically connected the emitter/sensor units 82(a,b,c) may be included with the circuit assembly and optical wave-guides used to transfer the sensing light to and from the monitoring point. The plurality of sensors may be connected to the processing unit individually, multiplexed, or aggregated to a sensor block or a combination of all three means.

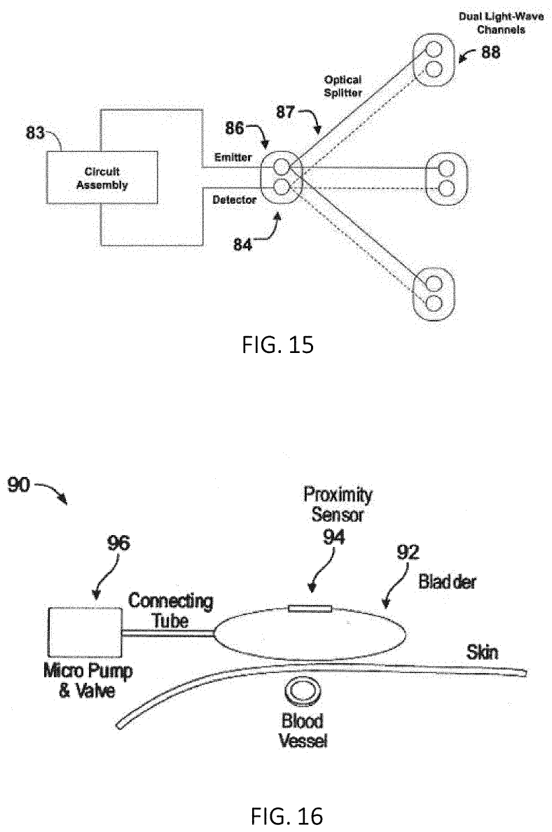

In the embodiments of FIGS. 14 and 15, the oximetry sensors include channels for directing light, e.g., lightwave guides or fiber optics 100, that are positioned to direct light to the measuring location on the user. In this manner, other components of the sensor can be spaced apart from the measuring location. For example, components of the oximetry sensor can be mounted on the circuit board along or in conjunction with use of light wave guides or optical fibers for skin contact. An advantage of the multiple light wave fibers over multiple sensors is that only one sensor pair (emitter 102 and detector 104) is needed and the smaller multiple light wave fibers can allow for more sensor data collection in a limited space. FIG. 14 provides for optical aggregation between the multiple sensors to a single detector 104 and emitter 102 and the multiple sensor points. Separate fibers may be used for the emitter path and the sensor path. FIG. 15 shows that alternately a single fiber/wave guide may be used from each sensor point and a suitable optical splitter 87 used to isolate the electronic emitter and detector/sensor paths.

With reference now to FIG. 16, a blood pressure monitor 90 is disposed in a headwear assembly (e.g., 10, 44, 59) in accordance with the present invention. The blood pressure monitor includes a pressurized bladder 92 disposed over a blood vessel of the user. Preferably, a blood vessel is near the surface of the user's skin, such as blood vessels in the temporal region of the scalp, such as the superficial temporal artery. A proximity sensor 94 is coupled to the bladder. In this arrangement, the volumetric change of the vessel is transferred to the bladder such that this volumetric change can be sensed by the sensor. Additionally, the bladder may be inflated and deflated using a small air pump 96 and bleed off valve.

Examples of effective sensors 94 include capacitive proximity sensors, e.g., which can translate displacement to an analog of the capacitance such as a voltage or digital count. Another example is a resistive band around the bladder, e.g., to translate circumference to resistance in a proportional manner. A pressure sensor can be attached to the bladder to measure pressure changes therein. An air pump 96 can be used to restrict the blood flow, periodically, to measure blood pressure, in a sphygmomanometer-type configuration.

Blood pressure monitor 90 can be located over a temporal region of the scalp or other area. Blood pressure sensor can include proximity sensors combined with small bladders to record volume displacement or capacitance sensors or stretch transducers to record displacement by way of voltage or resistance measurements, to measure the blood pressure of the user. The blood pressure monitor can be mounted on the substrate of the electronic strap/flex circuit (e.g., 12, 14, 60) and electronically coupled to the circuit board by a flexwire or other means. Multiple blood pressure monitors can be disposed strategically about the headwear assembly, to improve reliability by obtaining the strongest and most accurate reading from multiple measurements.

It should be appreciated from the foregoing that the present invention provides an exercise or athletic headwear assembly that measures physiological changes of a user during physical exercise, athletic activities, or other situations through the use of sensors. Through the placement of a headwear embodiment, the sensors will be placed in the preferred location on a user's head. The sensors measure the oxygen saturation, blood pressure, and pulse rate of a user. A thermistor can also be included to measure body temperature. The headwear assembly is capable of presenting data to the user through wireless transmission to an external smart mobile device, such as a smart phone, where it can be presented to the user by display, a recording or audio announcement on the device itself or through a wired or wireless listening device. The headwear assembly can include a wireless charger for charging a rechargeable battery via inductive charging.

Although the invention has been disclosed in detail with reference only to the exemplary embodiments, those skilled in the art will appreciate that various other embodiments can be provided without departing from the scope of the invention. Accordingly, the invention is defined by the claims set forth below.

What is claimed is:

1. An oximetry headwear assembly, comprising:
a headwear body having a front portion disposed proximate to the forehead of a user when worn, the headwear body comprising a headwear material;
an oximetry sensor having an emitter and a detector disposed in the front portion of the headwear body;
a circuit assembly having a processor module and a battery module, the circuit assembly electrically coupled to the oximetry sensor, the circuit assembly and the oximetry sensor configured to measure oxygen saturation and pulse rate of the user;
a waterproof casing enclosing the oximetry sensor and circuit assembly; and
a wireless battery charger removably coupled to the headwear body and disposed proximate to the battery module, the wireless battery charger configured to charge a rechargeable battery disposed within the battery module, via inductive charging.

2. The headwear assembly as defined in claim 1, wherein the oximetry sensor and circuit assembly are confined within the headwear body and enclosed by the headwear material, the headwear body provides an aperture in the front portion such that the oximetry sensor is aligned with the aperture proximate to the forehead, the aperture includes a transparent window enclosing the oximetry sensor, such that the oximetry sensor and circuit assembly are fully enclosed and inaccessible external to the headwear assembly, to provide enhanced waterproof protection.

3. The headwear assembly as defined in claim 1, further comprising a plurality of slip-resistant bands disposed about the headwear assembly, to be in contact with and about the head of the user wearing the headwear assembly, so as to prevent movement and maintain positioning of the headwear assembly.

4. The headwear assembly as defined in claim 3, wherein the plurality of slip-resistant bands comprise silicone embedded into the headwear material, so as to grip the skin and hair located on the head of the user.

5. The headwear assembly as defined in claim 1, wherein the circuit board assembly includes an LED driver electrically coupled to the emitter and a detector circuit operatively coupled to the detector, both the LED driver circuit and detector circuit are operatively coupled to the processor module, and the circuit assembly further includes a wireless transceiver operatively coupled to the processor module.

6. The headwear assembly as defined in claim 1, wherein the headwear material is a wicking, elasticized fabric configured to prevent movement and provide precise tension about the head of the user when worn, the elasticized fabric configured to be antimicrobial with silver impregnated particles disposed therein, enabling odor and stain resistance.

7. The headwear assembly as defined in claim 6, the headwear body includes an adjustable end having hook-and-loop attachment to maintain tension when worn.

8. The headwear assembly as defined in claim 1, further comprising a flexible substrate of unitary construction that is disposed in the front portion of the headwear body, the flexible substrate defines 1) a first section that houses the battery module, 2) a second section that houses the processor module, and 3) a third section that houses the oximetry sensor, the first and second sections defining a first neck region therebetween, the second and third sections defining a second neck region therebetween, the flexible substrate configured to conform to the shape of the head of the user wearing the headwear assembly.

9. The headwear assembly as defined in claim 8, wherein the flexible substrate further includes 1) a first stiffener mounted to the first section and on a side of the flexible substrate opposite to the battery module, 2) a second stiffener mounted to the second section and on a side of the flexible substrate opposite to the processor module, and 3) a third stiffener mounted to the third section and on a side of the flexible substrate opposite to the oximetry sensor, the first, second, and third stiffeners configured to protect the respective module and sensor.

10. The headwear assembly as defined in claim 9, wherein the flexible substrate includes wings extending from the first, second and third sections, such that the flexible substrate can be secured to the headwear assembly via said wings, such that the circuit assembly, oximetry sensor, and stiffeners are immobilized relative to the headwear assembly.

11. The headwear assembly as defined in claim 1, further comprising a charging pocket aligned with the battery module, so as to secure the wireless battery charger and enable inductive charging.

12. The headwear assembly as defined in claim 1, further comprising an upper light barrier, a lower light barrier, and one or more side light barriers for blocking ambient light to the oximetry sensor, the upper light barrier aligned with the upper forehead of the user wearing the headwear body, the lower and side light barriers aligned above and proximate to the eyebrows of the user wearing the headwear body, thereby providing light barrier material on all sides of the oximetry sensor.

13. The headwear assembly as defined in claim 1, further comprising a sensor cover disposed about the oximetry sensor, so as to prevent light from passing from the emitter to the detector directly.

14. The headwear assembly as defined in claim 1, further comprising a thermistor mounted adjacent to the oximetry sensor and confined within the waterproof casing, the thermistor configured to measure the temperature of the user wearing the headwear assembly.

15. The headwear assembly as defined in claim 14, further comprising a plurality of sensors, including blood pressure sensors disposed in prescribed locations on the headwear body to measure the blood pressure of the user when worn.

16. The headwear assembly as defined in claim 1, further comprising a plurality of circuit boards connected to each other in series via bridge connections, wherein the plurality of circuit boards are arranged in a linear fashion, and each of the plurality of circuit boards contain an electronic component.

17. An oximetry headwear assembly, comprising:
a headwear body having a front portion disposed proximate to the forehead of a user when worn, the headwear body comprising a headwear material;
a flexible substrate coupled to the front portion of the headwear body, the flexible substrate defining a plurality of sections coupled to one another in series, and a respective neck region of a plurality of neck regions disposed between each pair of adjacent sections of the plurality of sections, the flexible substrate is of unitary construction and configured to conform to the shape of the head of the user wearing the headwear assembly;
a battery module disposed on a first section of the plurality of sections, the battery module having a rechargeable battery disposed therein;
a processor module disposed on a second section of the plurality of sections, the processor module electrically coupled to the battery;
an oximetry sensor disposed on a third section of the plurality of sections, the oximetry sensor having an emitter and a detector, the oximetry sensor electrically coupled to the processor module and battery module, such that the processor module and oximetry sensor are configured to measure oxygen saturation and pulse rate of the user wearing the headwear assembly; and
a wireless battery charger removably coupled to the headwear body and disposed proximate to the battery module, the wireless battery charger configured to charge the rechargeable battery, via inductive charging.

18. The headwear assembly as defined in claim 17, wherein the flexible substrate is confined within the headwear body and enclosed by the headwear material, the headwear body provides an aperture in the front portion such that the oximetry sensor is aligned with the aperture proximate to the forehead, the aperture includes a transparent window enclosing the oximetry sensor, such that the oximetry sensor, processor module, and battery module are fully enclosed and inaccessible external to the headwear assembly.

19. The headwear assembly as defined in claim 18, further comprising a plurality of slip-resistant bands disposed about the headwear assembly, to be in contact with and about a head of the user wearing the headwear assembly, so as to prevent movement and maintain positioning of the headwear assembly.

20. The headwear assembly as defined in claim 19, further comprising a charging pocket aligned with the battery module, so as to secure the wireless battery charger and enable inductive charging.
